(19) United States
(12) Patent Application Publication
(10) Pub. No.: US 2016/0296703 A1
(43) Pub. Date: Oct. 13, 2016

(54) A Pharmaceutical Component-Mixing Delivery Assembly

(71) Applicant: 3P Innovation Limited, Warwickshire (GB)

(72) Inventors: Thomas Bailey, Warwick (GB); David Seaward, Warwick (GB)

(73) Assignee: 3P Innovation Limited, Warwick (GB)

(21) Appl. No.: 15/034,322

(22) PCT Filed: Nov. 5, 2014
(86) PCT No.: PCT/GB2014/053286

§ 371 Date: May 4, 2016

(30) Foreign Application Priority Data
Nov. 5, 2013 (GB) 1319467.5
Jul. 25, 2014 (GB) 1413208.8
Aug. 1, 2014 (GB) 1413715.2

(51) Int. Cl. A61M 5/24; A61M 5/28

(52) U.S. Cl. CPC A61M 5/2448; A61M 5/285; A61M 5/284; A61M 2005/2451

(57) ABSTRACT

There is described a pharmaceutical component-mixing delivery assembly comprising: a first cartridge and a second cartridge; the first and second cartridges being dimensioned so that they are movable relative to one another such that the first cartridge is slidable within the second cartridge from a pre-mixed position to a post-mixed position; the first cartridge comprising a body provided with an outlet end, the first cartridge being fitted with plunger and containing a first pharmaceutical component; the second cartridge comprising a body provided with an inlet end and an outlet end, the second cartridge containing a second pharmaceutical component; and wherein the first and second cartridges are separated by a non-invasive valve component.

### FIELD OF THE INVENTION

The present invention relates to a novel syringe suitable for mixing a pair of components.

More particularly, the invention relates to a novel syringe suitable for mixing a two component system, such as a solid medicament and a liquid, liquid/liquid components, viscous/non-viscous components, etc. The present invention also relates to methods related thereto.

### BACKGROUND TO THE INVENTION

Many medicaments are unsuitable for the long term storage as a liquid solution because they may be unstable when mixed into a solution and thus have a shorter shelf-life than other forms. Such medicaments may be reconstituted with a diluent or carrier, usually a liquid, prior to administration. Thus, liquid or solid medicaments may be stored in a sealed vial prior to reconstitution. Increasingly, prefilled syringes capable of separate storage of at least two different components are being used. Such syringes usually comprise a medicinal component stored in a first chamber and a dissolving or dispersing agent in a second chamber. The components are kept apart until use.

Dual chamber syringes have been developed which can suitably be used with powdered medicaments and a suitable liquid. Such dual chambers may be used for the administration of “biopharmaceuticals”, such as recombinant proteins; and the like.

One example of a commercially successful dual chamber syringe is the Vetter Lyo-Ject® dual chamber syringe, which is known to be used with recombinant anti-haemophilic factor. The recombinant anti-haemophilic factor is lyophilised in situ in the syringe. The Vetter syringe is described in U.S. Pat. No. 6,419,656 and comprises a tubular body extending along an axis; a plunger axially slidable in the body and a free piston slidable in the body forward of the plunger. The plunger subdivides the body into a front compartment, forward of the plunger, and a rear compartment between the plunger and the piston. The body is also provided with a bypass passage forward of the piston. In use, a lyophilised medicament is located in the front compartment and a liquid is located in the rear compartment. When the plunger is depressed and the second compartment is drawn alongside the bypass chamber, the liquid is able to flow into the first compartment and solubilise the medicament or form a suspension of the medicament.

However, one disadvantage of dual chamber syringes, such as the Vetter Lyo-Ject® syringe, is that it is expensive to manufacture. Also producing a lyophilised powdered medicament in situ in the syringe is difficult and does not lend itself to rapid and large scale manufacturing techniques.

Other dual chamber syringes are known. For example, U.S. Pat. No. 5,281,198 describes a syringe assembly suitable for multiple pharmaceutical components, such as a lyophilized medicament component and a diluent component contained within first and second cartridges fitted with a movable piston. The second cartridge is forced into the interior of the first cartridge causing a spike or needle assembly to couple the two cartridges and causing the contents of the first cartridge to be driven into the second cartridge, thereby mixing them. A ratchet plunger is used to drive a second piston, the distance that the ratchet plunger moves determining the dose administered.

However, one disadvantage with such devices is that small particles may be generated when the spike or needle assembly penetrates or pierces a septum dividing a first and second cartridge. Furthermore, the user may experience considerable resistance or friction in pushing the plunger to pierce the septum. It is also important that the sterility of the syringe is maintained until the device is ready to be used.

### SUMMARY OF THE INVENTION

We have now found a device that overcomes or mitigates the problems associated with the prior art devices. More particularly, we have found a novel dual syringe assembly which avoids the need to pierce a septum. Furthermore, the novel device of the present invention can suitably utilise conventional standard syringe components, avoiding the need for a bypass chamber to be built into the body of a syringe.

Thus, according to a first aspect of the present invention there is provided a pharmaceutical component-mixing delivery assembly comprising:
a first cartridge and a second cartridge;
the first and second cartridges being dimensioned so that they are movable relative to one another such that the first cartridge is slidable within the second cartridge from a pre-mixed position to a post-mixed position;
the first cartridge comprising a body provided with an outlet end, the first cartridge being fitted with plunger and containing a first pharmaceutical component;
the second cartridge comprising a body provided with an inlet end and an outlet end, the second cartridge containing a second pharmaceutical component; and
wherein the first and second cartridges are separated by a non-invasive valve component.

In a particular embodiment the non-invasive valve component, e.g. comprising a piston, is provided with a terminal rib, that is, a circumferential rib located at the end of the non-invasive valve component nearest to the open end of the second cartridge. The inclusion of a terminal rib is advantageous in that, inter alia, it allows the sterility of the delivery assembly to be maintained when the device is primed before injection.

Furthermore, the non-invasive valve component, e.g. a piston, may be generally elongated. The region of the second cartridge that contains the second pharmaceutical component will generally comprise an aseptic mixing chamber, whilst the inner wall of the second cartridge outside of the mixing chamber and beyond the terminal rib of the piston, will generally be non-aseptic. In use, as the plunger of the delivery assembly is depressed and the piston moves towards the inlet end of the second cartridge, the mixing chamber essentially expands longitudinally. Thus, the length of the piston is desirably chosen so that as the piston moves, the mixing chamber never comes into contact with the non-aseptic walls of the second cartridge and the sterility of the mixing chamber is maintained at all times. Therefore, the length of the piston will be dictated by how far back along the second cartridge it has to move, which will in turn be dictated by the volume required for the mixing chamber.

In a particular aspect of the invention the non-invasive valve component may comprise a ball valve, a flow through stopper component or a T-shaped stopper component.

The pharmaceutical component-mixing delivery assembly will generally comprise a syringe assembly. Thus, the pharmaceutical component-mixing delivery assembly hereinbefore described may include a needle, e.g. a hypodermic needle, attached to the outlet end of the second cartridge.

One of the first and second pharmaceutical components may comprise a solid therapeutically active agent and the other is a liquid capable of solubilising or forming a suspension with the solid therapeutically active agent. In a particular aspect of the invention the first pharmaceutical component comprises a liquid and the second pharmaceutical component comprises a solid therapeutically active agent. However, it will be understood by the person skilled in the art that the first and second pharmaceutical components may comprise liquid/liquid components, viscous/non-viscous components, etc.

The use of a non-invasive valve as hereinbefore described is advantageous in that, inter alia, conventional syringe bodies and/or plungers may be utilised, significantly reducing the expense of the syringe assembly as a whole. It also avoids, for example, the need for an elastomeric septum.

In one aspect of the invention the non-invasive valve component may comprise a ball valve. Such a ball valve component may comprise one or more balls located within the outlet of the first cartridge, such that the ball(s) acts to seal the first cartridge. The ball valve component includes a substantially hollow apertured piston suitable for receiving the ball from the first cartridge.

More specifically, the ball valve component may comprise a substantially hollow apertured piston with a first end adjacent the outlet end of the first cartridge and a second end adjacent the inlet end of the second cartridge. The hollow apertured piston will comprise side walls which generally form a seal against the inner walls of the second cartridge.

The first end of the piston may comprise means for locating the outlet end of the first cartridge. For example, such means may comprise a substantially circumferential ridge. The second end of the piston may comprise one or more apertures which, when in use, are dimensioned to allow fluid to flow.

A single ball or a plurality, e.g. two or three balls, may be located within the outlet of the first cartridge, such that the ball(s) acts to seal the first cartridge.

In use, when the plunger is depressed within the first cartridge the fluid pressure will force the ball valve to open allowing fluid to flow from the first cartridge to the second cartridge enabling mixing to occur.

In another aspect of the invention the non-invasive valve component may comprise a ball valve as hereinbefore described. However, the second end of the piston may comprise a separate plug component which is provided with one or more apertures which, when in use, are dimensioned to allow fluid to flow. In addition, the plug component may be provided with means for displacing the ball from the outlet of the first cartridge allowing fluid to flow from the first cartridge to the second cartridge enabling mixing to occur. Thus, in accordance with this aspect of the invention there is provided a pharmaceutical component-mixing delivery assembly as hereinbefore described wherein the ball valve component includes a plug component which is provided with one or more apertures and which is provided with means for displacing the ball from the outlet of the first cartridge. One example of such displacement means is a plug provided with a protruding spigot.

In another aspect of the invention the non-invasive valve component may comprise a flow through stopper component. The flow through stopper component may comprise a stopper which is provided with conduit means. Said conduit means may comprise an internal conduit within the stopper or alternatively, may comprise an external conduit, that is, a conduit between the walls of the piston and the stopper.

One example of a flow through stopper component with an internal conduit is a T-valve. Such a T-valve component may comprise a substantially hollow apertured piston with a first end adjacent the outlet end of the first cartridge and a second end adjacent the inlet end of the second cartridge. The hollow apertured piston will comprise side walls which generally form a seal against the inner walls of the second cartridge as hereinbefore described.

The first end of the piston may comprise means for locating the outlet end of the first cartridge. For example, such means may comprise a substantially circumferential ridge. The piston also houses a stopper which has a flow through capability and which seals the outlet end of the first cartridge. Said stopper may comprise an internal conduit which is substantially coaxial with the longitudinal axis of the first cartridge, but which is provided with one or more branches which seal against the side walls of the piston.

In use, when the plunger is depressed within the first cartridge the fluid pressure will displace the flow through stopper such that the one or more branches of the conduit are no longer sealed against the side walls of the piston allowing fluid to flow from the first cartridge to the second cartridge enabling mixing to occur. Optionally, a second stopper may be provided adjacent the inlet of the second cartridge. The second stopper may also be provided with one or more branched conduits, which act in a substantially similar manner to the first.

In any of the embodiments described herein the side walls of the non-invasive valve component may be provided with one or more external ribs to improve the seal formed between the non-invasive valve component and the second cartridge. Thus, for example, when the non-invasive valve component comprises a piston, the piston may be ribbed, i.e. is provided with one or more external ribs, to improve the seal formed between the piston and the inner walls of the second cartridge. Preferably the piston is provided with one or more circumferential ribs. It is especially preferred for the piston to be provided with a plurality of circumferential ribs, e.g. 2, 3 or 4 ribs.

An alternative example of a flow through stopper component with an external conduit is an “annular valve”. Although an “annular valve” possesses a similar functionality to the aforementioned T-valve, the “annular valve” stopper itself does not comprise an internal conduit.

Adjacent the first cartridge the internal walls of the piston are configured to form a snug fit with the stopper. In this first position, the stopper seals the first cartridge. The piston also provides a second position wherein the stopper forms a loose fit, such a conduit external to the stopper is formed between the stopper and the piston, such that fluid may pass through the conduit, between the walls of the piston and the stopper.

Thus, according to this aspect of the invention there is provided a pharmaceutical component-mixing delivery assembly as hereinbefore described wherein the “annular valve” comprises a piston configured to form a snug fit with a stopper which seals the first cartridge; and wherein the piston provides a second position wherein the stopper forms a loose fit, such that fluid may pass between the walls of the piston and the stopper.

The use of a T-valve is advantageous, inter alia, in that:
It provides guidance for the piston in the hollow stopper (the plug cannot tilt)
Aids assembly—physical stop
Prevents the piston being accidently drawn back into the syringe
Ribs provide enhanced sealing in the neck of the of the cartridge
The larger diameter part is held by the hollow stopper which provides additional retention of the piston

According to a further aspect of the present invention there is provided a method of operating a pharmaceutical component-mixing delivery assembly holding first and second pharmaceutical components; said delivery assembly comprising:
a first cartridge and a second cartridge;
the first and second cartridges being dimensioned so that they are movable relative to one another such that the first cartridge is slidable within the second cartridge from a pre-mixed position to a post-mixed position;
the first cartridge comprising a body provided with an outlet end, the first cartridge being fitted with plunger and containing a first pharmaceutical component;
the second cartridge comprising a body provided with an inlet end and an outlet end, the second cartridge containing a second pharmaceutical component; and
wherein the first and second cartridges are separated by a non-invasive valve component;
   said method comprising pressing the plunger axially into the first cartridge to an intermediate position thus pressurizing the first cartridge and opening the non-invasive valve component, causing the first pharmaceutical component to flow through the valve and into the second cartridge to mix with the second pharmaceutical component.

According to a yet further aspect of the present invention there is provided a method of administering a therapeutically active agent to a patient which comprises operating a pharmaceutical component-mixing delivery assembly holding first and second pharmaceutical components; said delivery assembly comprising:
a first cartridge and a second cartridge;
the first and second cartridges being dimensioned so that they are movable relative to one another such that the first cartridge is slidable within the second cartridge from a pre-mixed position to a post-mixed position;
the first cartridge comprising a body provided with an outlet end, the first cartridge being fitted with plunger and containing a first pharmaceutical component;
the second cartridge comprising a body provided with an inlet end and an outlet end, the second cartridge containing a second pharmaceutical component; and
wherein the first and second cartridges are separated by a non-invasive valve component;
   said method comprising the steps of:
(i) pressing the plunger axially into the first cartridge to an intermediate position thus pressurizing the first cartridge and opening the a non-invasive valve component, causing the first pharmaceutical component to flow through the valve and into the second cartridge to mix with the second pharmaceutical component;
(ii) after the first and second pharmaceutical components have mixed, administering the mixed pharmaceutical components to a patient.

According to a particular aspect of the method of the invention the non-invasive valve component is provided with a terminal rib to improve the seal formed between the non-invasive valve component and the second cartridge.

The pharmaceutical component-mixing delivery assembly will generally comprise a syringe assembly. Thus, the method of administering a therapeutically active agent to a patient will generally comprises an additional step of attaching a needle, e.g. a hypodermic needle, to the outlet end of the second cartridge, followed by administering the mixed pharmaceutical components to a patient.

In the aforementioned methods preferably one of the first and second pharmaceutical components comprises a solid therapeutically active agent and the other is a liquid capable of solubilising or forming a suspension with the solid therapeutically active agent. In a particular aspect of the invention the first pharmaceutical component comprises a liquid and the second pharmaceutical component comprises a solid therapeutically active agent. Alternatively, the first and second pharmaceutical components may comprise liquid/liquid components, viscous/non-viscous components, etc.

In another embodiment of the present invention resistance or friction may be minimised for the user by providing a plunger with an external screw thread. In this particular embodiment the plunger is desirably threaded along a predetermined portion of its length. It is within the scope of the present invention for the plunger to be threaded along the whole of its length, however, it is preferred that the plunger is only partially threaded, i.e. threaded along only a portion of its length. Thus, the user may screw the plunger to bring the contents of the first and second cartridges into contact, since this is generally where the user experiences most resistance. However, the remainder of the shaft of the plunger may desirably be unthreaded so that a conventional push action may be used for administration of the medicament solution to a patient.

The use of a threaded, or partially threaded, plunger as hereinbefore described may also be advantageous in that it may be used to provide a sealed system, improving sterility of the delivery device. Thus, a frangible seal may be provided around the shaft of the plunger, for example, within the body of the first cartridge, such that twisting of the plunger by the user can cause the frangible seal to break.

The use of a threaded plunger may suitably be used with any of the aforementioned syringe embodiments hereinbefore described. However, it will be understood by the person skilled in the art that such a threaded plunger arrangement is novel per se and may therefore be suitably applied to a conventionally known invasive valve component, such as a needle piercing an elastomeric septum. Desirably, the pharmaceutical component-mixing delivery assembly according to this aspect of the invention will generally comprise a sealed unit, e.g. an outer casing which house the first and second cartridges.

A particular advantage of the use of a threaded plunger is that a seal may be placed around the end of the first cartridge by, i.e. between the first cartridge and the outer casing.

Thus, according to this further aspect of the present invention there is provided a pharmaceutical component-mixing delivery assembly comprising:
an outer casing is provided which houses a first and a second cartridge;
the first and second cartridges being dimensioned so that they are movable relative to one another such that the first cartridge is slidable within the second cartridge from a pre-mixed position to a post-mixed position;
the first cartridge comprising a body provided with an outlet end, the first cartridge being fitted with an at least partially threaded plunger the end of which protrudes from the outer casing, said first cartridge containing a first pharmaceutical component;
the second cartridge comprising a body provided with an inlet end and an outlet end, the second cartridge containing a second pharmaceutical component;
wherein the first and second cartridges are separated by a valve component; and
a frangible seal is provided between the outer casing and the first cartridge.

In use, when the at least partially threaded plunger is twisted the frangible seal is broken; then as the at least partially threaded plunger and the cartridge are pushed towards the second cartridge the first cartridge disengages from the seal. The use of a frangible seal enables the device to remain sterile during storage, whilst the disengagement of the frangible seal from the first cartridge reduces friction and lowers force required to complete the administration of the therapeutically active agent.

According to this aspect of the invention the valve component may comprise a non-invasive valve as hereinbefore described or a conventionally known valve, such as a needle valve or a bypass valve.

In a further aspect of the invention there is provided a method of administering a therapeutically active agent to a patient which comprises operating a pharmaceutical component-mixing delivery assembly holding first and second pharmaceutical components; said delivery assembly comprising:
an outer casing is provided which houses a first and a second cartridge;
the first and second cartridges being dimensioned so that they are movable relative to one another such that the first cartridge is slidable within the second cartridge from a pre-mixed position to a post-mixed position;
the first cartridge comprising a body provided with an outlet end, the first cartridge being fitted with an at least partially threaded plunger the end of which protrudes from the outer casing, said first cartridge containing a first pharmaceutical component;
the second cartridge comprising a body provided with an inlet end and an outlet end, the second cartridge containing a second pharmaceutical component;
wherein the first and second cartridges are separated by a valve component; and
a frangible seal is provided between the outer casing and the first cartridge.

The method of operation according to this aspect of the invention will generally comprise first turning the at least partially threaded plunger causing the frangible seal to break and disengage from the first cartridge and subsequently pressing the plunger to administer the mixed pharmaceutical components to a patient.

The term “non-invasive valve” will be understood by the person skilled in the art. However, for the avoidance of doubt, the term should be construed so as not to include a component wherein, for example, a needle is utilised to pierce a septum and the like. Furthermore, the term “non-invasive valve component” should be construed as meaning that the valve is made up entirely of the component, i.e. it does not utilise the wall of the syringe or cartridge (which is distinct from, for example, the Vetter syringe described in U.S. Pat. No. 6,419,656).

According to a yet further aspect of the invention there is provided a kit suitable for mixing and delivery of pharmaceutical components, said kit comprising:
a first cartridge and a second cartridge;
the first and second cartridges being dimensioned so that when placed together they are movable relative to one another such that the first cartridge is slidable within the second cartridge from a pre-mixed position to a post-mixed position;
the first cartridge comprising a body provided with an outlet end, the first cartridge being fitted with plunger and containing a first pharmaceutical component;
the second cartridge comprising a body provided with an inlet end and an outlet end, the second cartridge containing a second pharmaceutical component; and
wherein when the first and second cartridges are placed together they are separated by a non-invasive valve component.

There is also provided a kit suitable for mixing and delivery of pharmaceutical components, said kit comprising:
an outer casing which houses a first and a second cartridge;
the first and second cartridges being dimensioned so that they are movable relative to one another such that the first cartridge is slidable within the second cartridge from a pre-mixed position to a post-mixed position;
the first cartridge comprising a body provided with an outlet end, the first cartridge being fitted with an at least partially threaded plunger the end of which protrudes from the outer casing, said first cartridge containing a first pharmaceutical component;
the second cartridge comprising a body provided with an inlet end and an outlet end, the second cartridge containing a second pharmaceutical component;
wherein the first and second cartridges are separated by a valve component; and
a frangible seal is provided between the outer casing and the first cartridge.

The invention further provides the use of a pharmaceutical component-mixing delivery assembly comprising:
a first cartridge and a second cartridge;
the first and second cartridges being dimensioned so that they are movable relative to one another such that the first cartridge is slidable within the second cartridge from a pre-mixed position to a post-mixed position;
the first cartridge comprising a body provided with an outlet end, the first cartridge being fitted with plunger and containing a first pharmaceutical component;
the second cartridge comprising a body provided with an inlet end and an outlet end, the second cartridge containing a second pharmaceutical component; and
wherein the first and second cartridges are separated by a non-invasive valve component;
for the mixing and delivery of a first and second pharmaceutical component.

According to this aspect of the invention the non-invasive valve component is provided with a terminal rib to improve the seal formed between the non-invasive valve component and the second cartridge.

The key advantages of the dual cartridge pharmaceutical component-mixing delivery assembly of the present invention can be summarised as follows:
Minimal air present in the reconstitution process
Use of many standard components
Diluent, Powder cartridges separate (processing in parallel, separate lines)
Barrier properties
Visible reconstitution
Low waste/deadspace
Minimal number of components in contact with the powder and diluent
Less Air—removes priming step and enables auto-injection
Minimal Stability/Drug interaction issues—
Lower cost
Plastic/glass combinations can be used
Customisable for different pharmaceuticals
Bulk lyophilisation of the solid component will be possible
Reduced formulation burden
Use of many conventional components
Bulk testing before filling would be possible
Multiple powders can be filled

The invention will now be illustrated by way of example only and with reference to the accompanying figures in which:

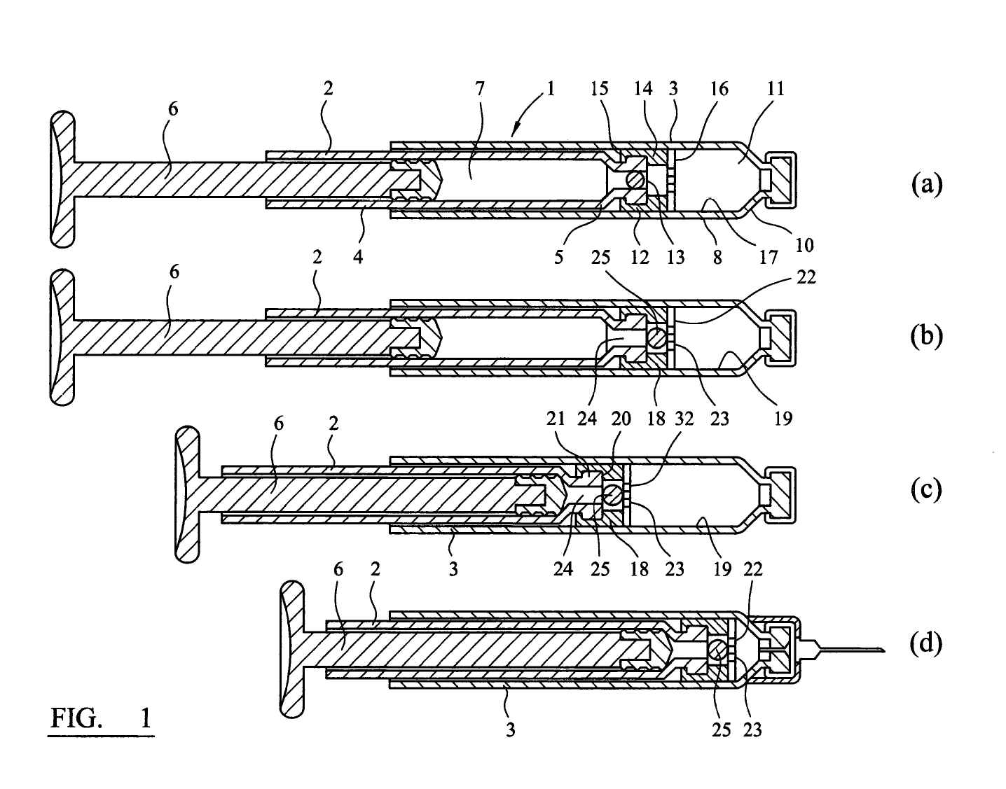

FIGS. 1 (a) to (d) are cross-sectional views of a pharmaceutical component-mixing delivery assembly comprising a ball valve component;

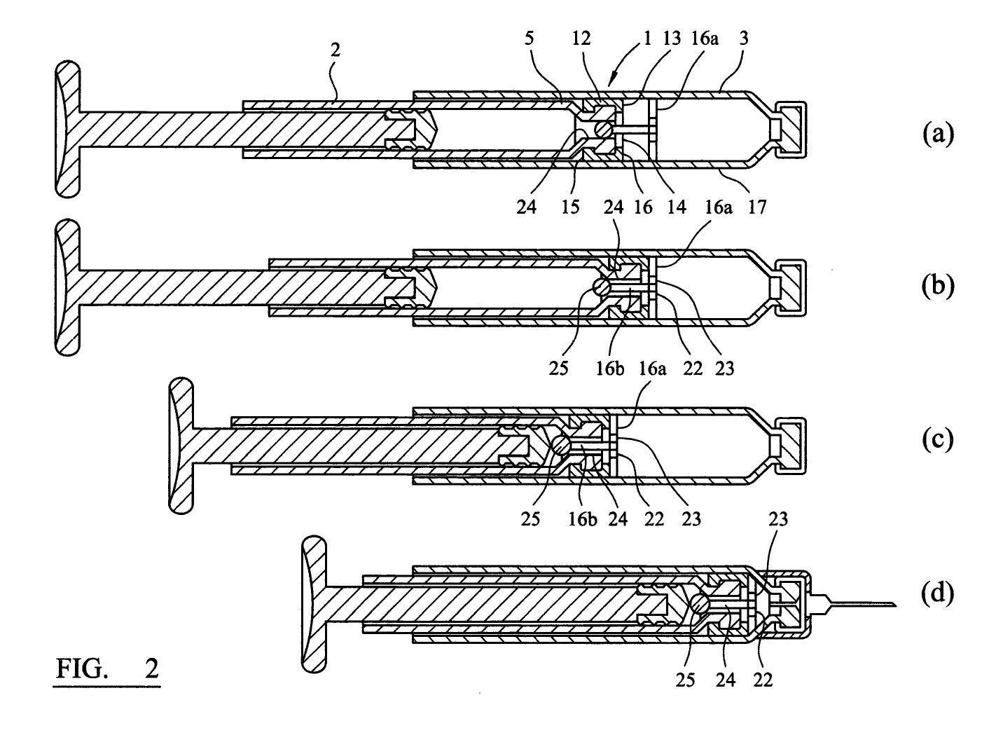

FIGS. 2 (a) to (d) are cross-sectional views of a pharmaceutical component-mixing delivery assembly comprising a ball valve component provided with ball displacement means;

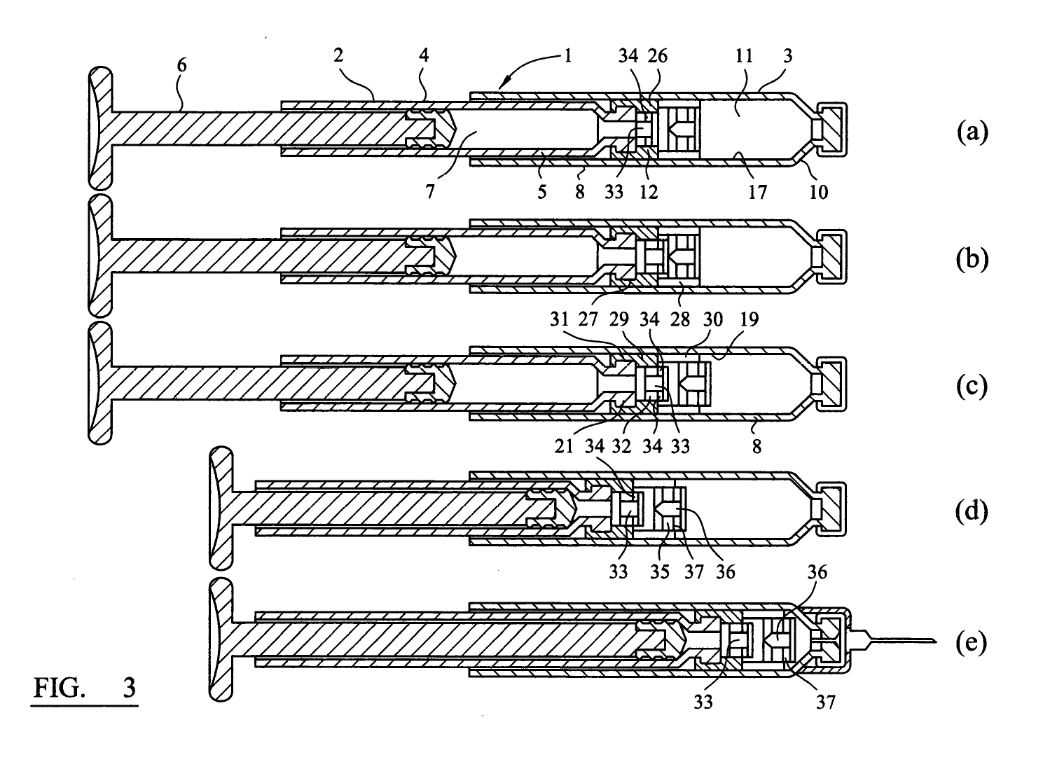

FIGS. 3 (a) to (e) are cross-sectional views of a pharmaceutical component-mixing delivery assembly comprising a T-valve component;

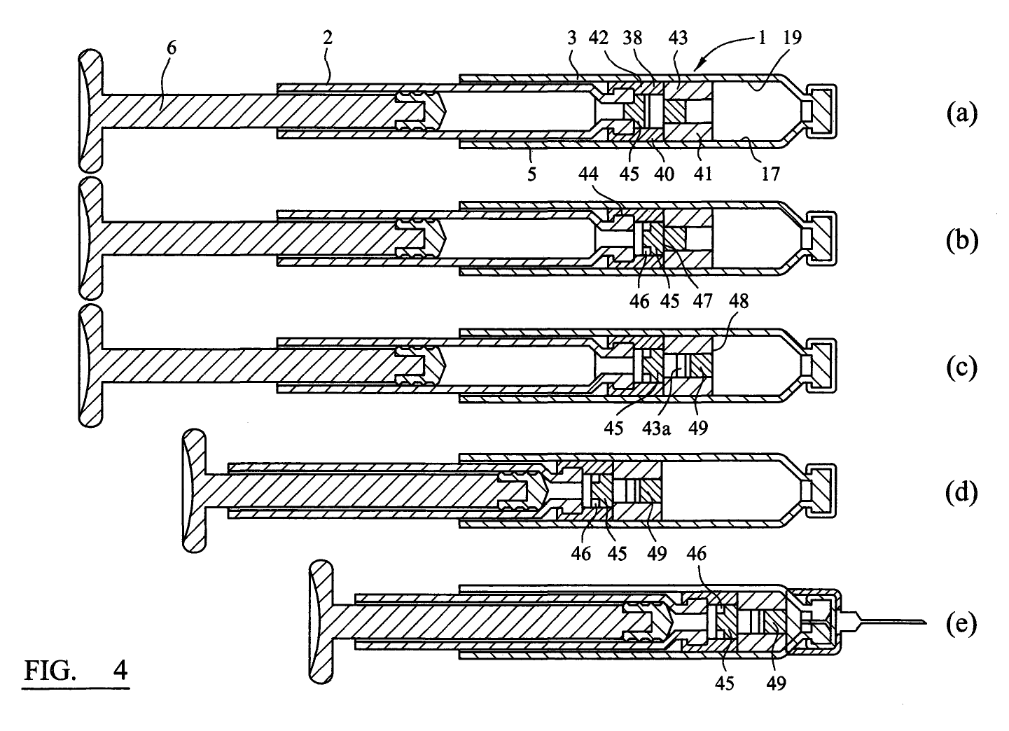

FIGS. 4 (a) to (e) are cross-sectional views of a pharmaceutical component-mixing delivery assembly comprising an “annular valve” component;

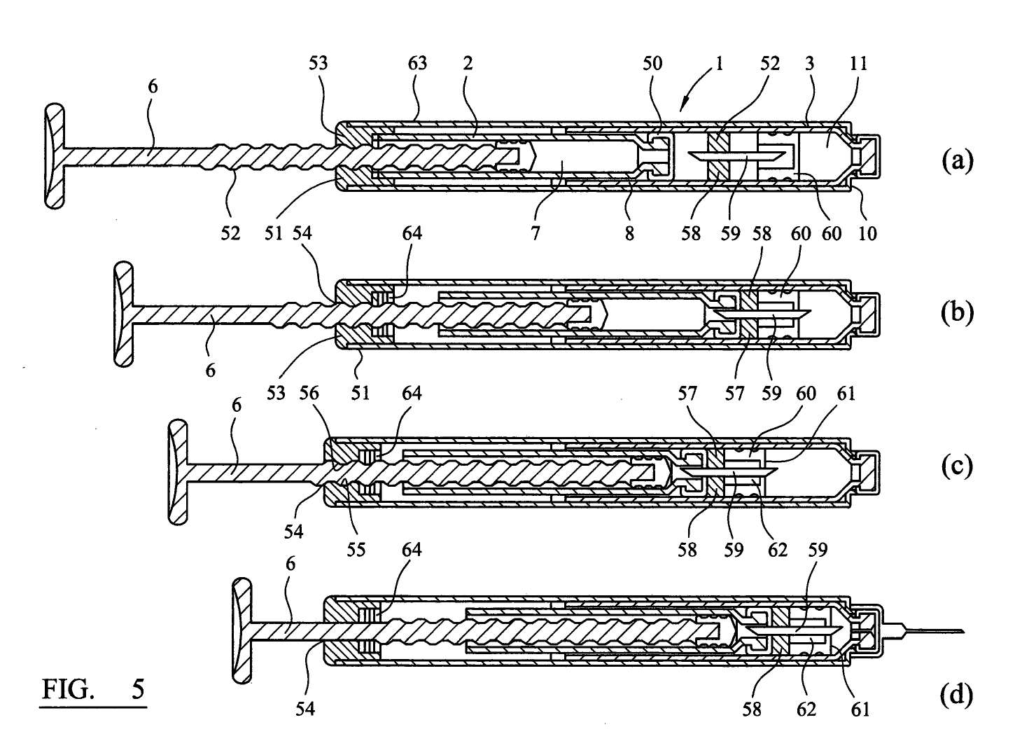

FIGS. 5 (a) to (d) are cross-sectional views of a sealed pharmaceutical component-mixing delivery assembly comprising a partially threaded plunger and “needle valve” component;

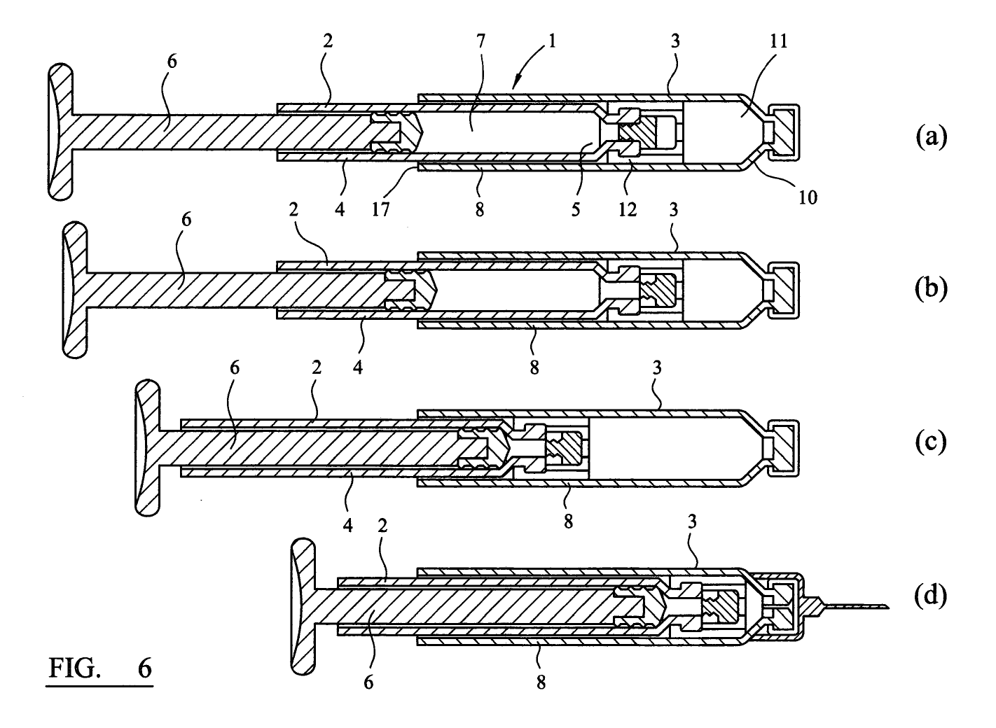

FIGS. 6 (a) to (d) are cross-sectional views of a pharmaceutical component-mixing delivery assembly comprising a spigot plug component;

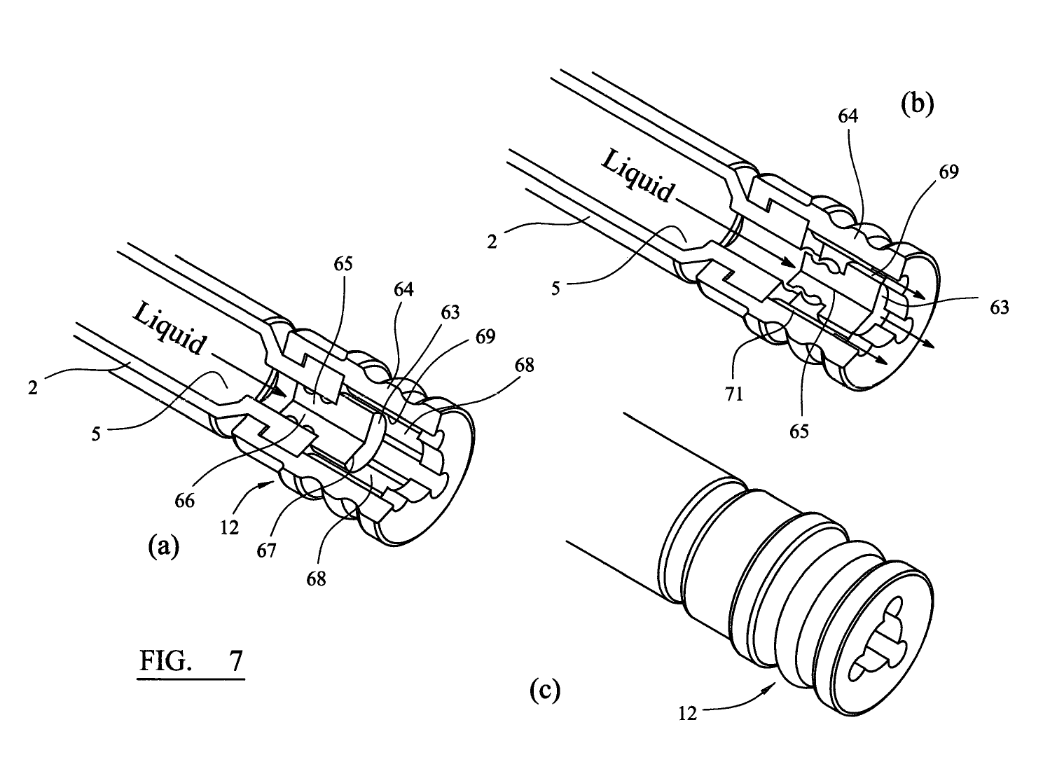

FIGS. 7 (a) and 7 (b) are cutaway perspective views of a pharmaceutical component-mixing delivery assembly comprising a spigot plug component of FIG. 6; and

FIG. 7 (c) is a perspective view of the delivery assembly of FIG. 6;

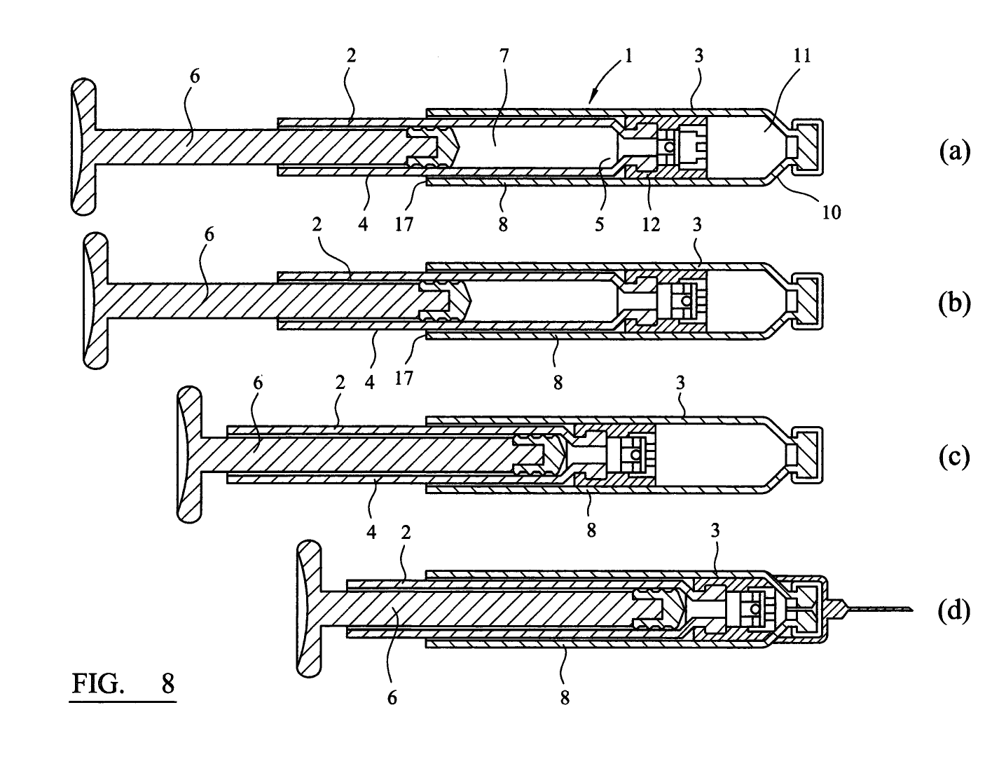

FIGS. 8 (a) to (d) are cross-sectional views of a pharmaceutical component-mixing delivery assembly comprising a hollow plug component;

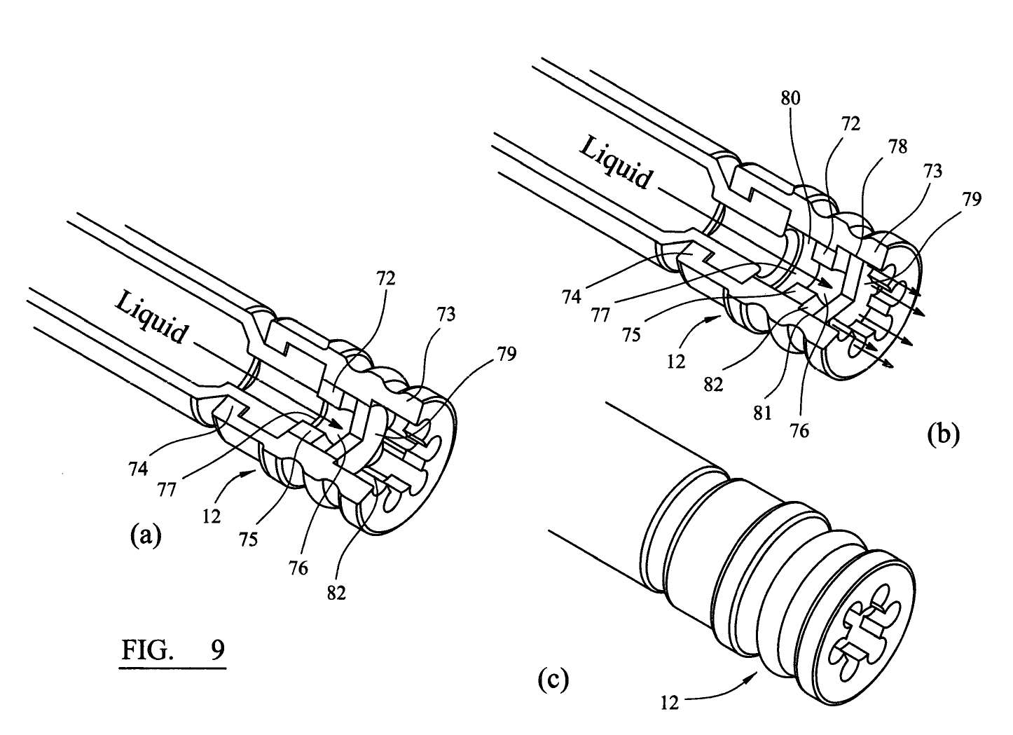

FIGS. 9 (a) and 9 (b) are cutaway perspective views of a pharmaceutical component-mixing delivery assembly comprising a hollow plug component of FIG. 8; FIG. 9 (c) is a perspective view of the delivery assembly of FIG. 8;

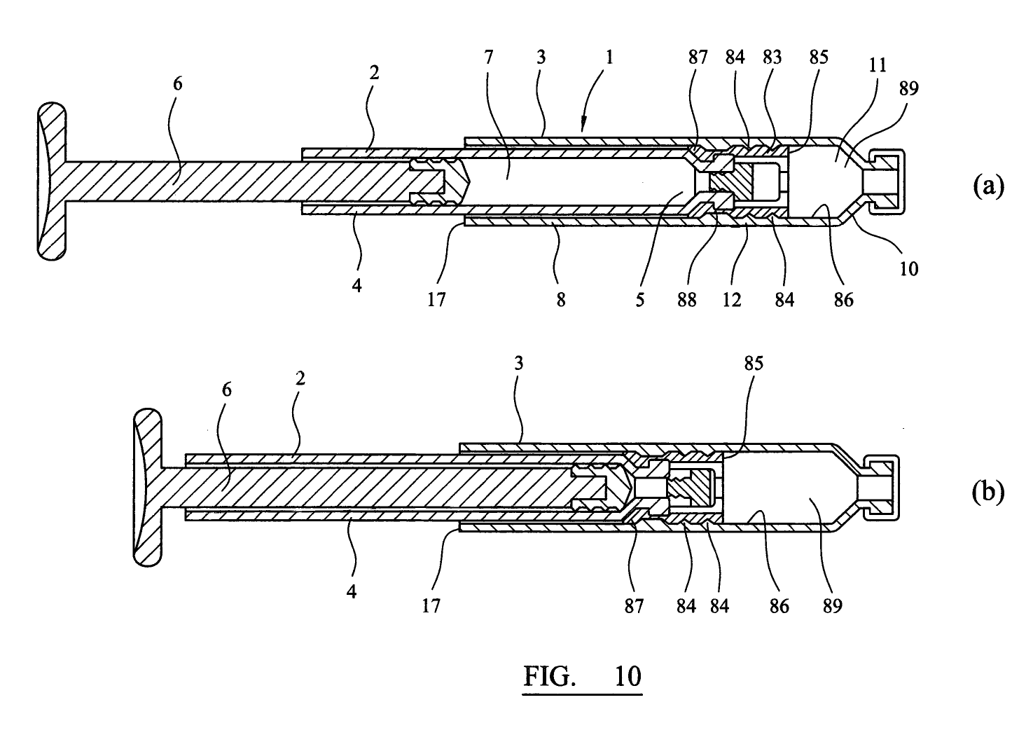

FIGS. 10 (a) and (b) are cross-sectional views of a pharmaceutical component-mixing delivery assembly comprising a ribbed non-invasive valve component;

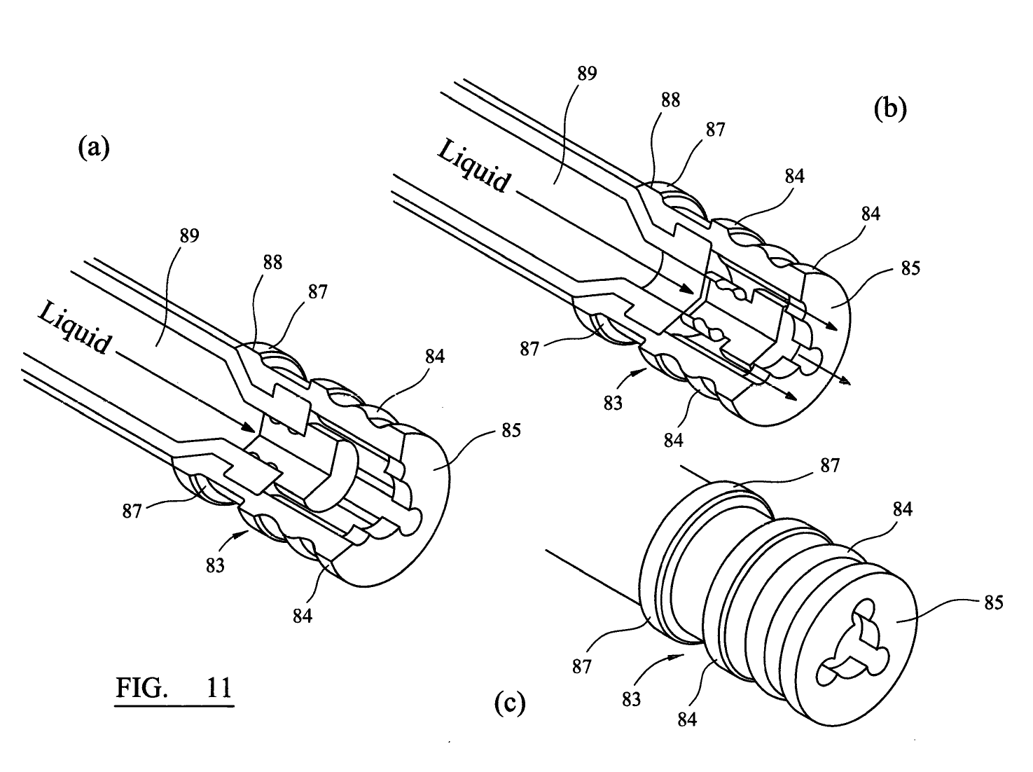

FIGS. 11 (a) and 11 (b) are cutaway perspective views of a pharmaceutical component-mixing delivery assembly comprising ribbed non-invasive valve component of FIG. 10; and

FIG. 11 (c) is a perspective view of the delivery assembly of FIG. 10.

Referring to FIGS. 1 (a) to (d) a pharmaceutical component-mixing delivery assembly (1) comprises a first cartridge (2) and a second cartridge (3), the first cartridge (2) being dimensioned so that it is slidable within the second cartridge (3) from a pre-mixed position to a post-mixed position. The first cartridge (2) comprises a body (4) provided with an outlet end (5) and a plunger (6). The first cartridge (2) contains a first pharmaceutical component (7). The second cartridge (3) comprises a body (8) provided with an inlet end (17) and an outlet end (10). The second cartridge (3) contains a second pharmaceutical component (11). A non-invasive valve component (12) separates the first and second cartridges (2) and (3).

The non-invasive valve component (12) comprises a ball valve (13) which consists of a hollow piston (14) with a first end (15) adjacent the outlet end (5) of the first cartridge (2) and a second end (16) adjacent the inlet end (17) of the second cartridge (3). The hollow piston (14) comprises a side wall (18) which generally forms a seal against the inner surface (19) of the body (8) of the second cartridge (3).

The first end (15) of the hollow piston (14) is provided with a circumferential recess (20) suitable for locating the outlet end (5) of the first cartridge (2). In the embodiment shown the outlet end (5) of the first cartridge (2) is provided with a circumferential shoulder (21) adapted to fit into the circumferential recess (20) of the first end (15) of the hollow piston (14). The second end (16) of the hollow piston (14) is provided with a pair of apertures (22) and (23). The outlet end (5) of the first cartridge (2) is provided with an orifice (24) within which sits a ball (25) which acts to seal the first cartridge (2).

In use, when the plunger (6) is depressed within the first cartridge (2) the increase in pressure will force the ball (25) out of the orifice (24), which causes the valve component (12) to open allowing the first pharmaceutical component (7), usually a fluid to flow from the first cartridge (2) through orifice (24), around ball (25), through apertures (22) and (23) and into the second cartridge (3) where it mixes with the second pharmaceutical component (11).

Referring to FIGS. 2 (a) to (d) a pharmaceutical component-mixing delivery assembly (1) comprises a first cartridge (2) and a second cartridge (3) as hereinbefore described with reference to FIGS. 1 (a) to (d). The non-invasive valve component (12) comprises a ball valve (13) which consists of a hollow piston (14) with a first end (15) adjacent the outlet end (5) of the first cartridge (2) and a second end (16) adjacent the inlet end (17) of the second cartridge (3) as hereinbefore described.

However, the second end (16) of the hollow piston (14) comprises a separate plug component (16a) which is provided with a pair of apertures (22) and (23). In addition, the plug component (16a) is provided with a substantially central spigot (16b) which protrudes from plug component (16a) towards the outlet end (5) of the first cartridge (2).

In use, when the plunger (6) is depressed within the first cartridge (2) the first cartridge (2) slides towards the plug component (16a). When the spigot (16b) comes into contact with the ball (25), the ball (25) is displaced from orifice (24), which causes the valve component (12) to open allowing the first pharmaceutical component (7), usually a fluid to flow from the first cartridge (2) through orifice (24), around ball (25), through apertures (22) and (23) and into the second cartridge (3) where it mixes with the second pharmaceutical component (11).

Referring to FIGS. 3 (a) to (e), a pharmaceutical component-mixing delivery assembly (1) comprises a first cartridge (2) and a second cartridge (3), the first cartridge (2) being dimensioned so that it is slidable within the second cartridge (3) from a pre-mixed position to a post-mixed position. The first cartridge (2) comprises a body (4) provided with an outlet end (5) and a plunger (6). The first cartridge (2) contains a first pharmaceutical component (7). The second cartridge (3) comprises a body (8) provided with an inlet end (17) and an outlet end (10). The second cartridge (3) contains a second pharmaceutical component (11). A non-invasive valve component (12) separates the first and second cartridges (2) and (3).

The non-invasive valve component (12) comprises a T-valve component (26). The T-valve component (26) consists of a pair of hollow pistons (27) and (28). A first hollow piston (27) is adjacent the outlet end (5) of the first cartridge (2) and a second hollow piston (28) is adjacent the inlet end (17) of the second cartridge (3). Each of the hollow pistons (27) and (28) comprises a side wall (29) and (30) respectively which generally forms a seal against the inner surface (19) of the body (8) of the second cartridge (3).

The first hollow piston (27) is provided with a circumferential recess (31) suitable for locating the outlet end (5) of the first cartridge (2). In the embodiment shown the outlet end (5) of the first cartridge (2) is provided with a circumferential shoulder (21) adapted to fit into the circumferential recess (31) of the first hollow piston (27). The first hollow piston (27) is fitted with a stopper component (32) with an internal conduit (33) which is substantially coaxial with the longitudinal axis of the first cartridge (2). The internal conduit (33) is provided with a branch (34) which seals against the side wall (29) of the hollow piston (27).

The second hollow piston (28) is fitted with a second stopper component (35) with an internal conduit (36) which is substantially coaxial with the longitudinal axis of the first cartridge (2). The internal conduit (36) is provided with a branch (37) which seals against the side wall (30) of the hollow piston (28).

In use, when the plunger (6) is depressed within the first cartridge (2) the increase in pressure will force the stopper component (32) to protrude out of the first hollow piston (27). This movement causes the branched conduit (34) to be exposed and lose its seals against the side wall (29) of the hollow piston (27) allowing the first pharmaceutical component (7), usually a fluid, to flow from the first cartridge (2) through internal conduit (33) and branched conduit (34) into the second hollow piston (28). Continued depression of the plunger (6) causes the second stopper component (35) to protrude out of the second hollow piston (28). This movement causes the branched conduit (37) to be exposed and lose its seal against the side wall (30) of the second hollow piston (28) allowing the first pharmaceutical component (7) to flow from the first cartridge (2) and first hollow piston (27) through internal conduit (36) and branched conduit (37) into the second cartridge (3) where it mixes with the second pharmaceutical component (11).

Referring to FIGS. 4 (a) to (e), a pharmaceutical component-mixing delivery assembly (1) comprises first and second cartridges (2) and (3) as hereinbefore described.

An annular valve component (38) comprises a round stopper component (39) with a conduit external to the stopper. This is hereinafter referred to an “annular valve”.

The “annular valve” component (38) consists of a pair of hollow pistons (40) and (41). A first hollow piston (40) is adjacent the outlet end (5) of the first cartridge (2) and a second hollow piston (41) is adjacent the inlet end (17) of the second cartridge (3). Each of the hollow pistons (40) and (41) comprises a side wall (42) and (43) respectively which generally forms a seal against the inner surface (19) of the body (8) of the second cartridge (3).

The first hollow piston (40) is provided with a circumferential recess (44) suitable for locating the outlet end (5) of the first cartridge (2) The first hollow piston (40) is fitted with a first stopper component (45) with a circumferential shoulder (46) adapted to fit into the outlet end (5) of the first cartridge (2) and seal it.

The first hollow piston (40) comprises a star shaped orifice (47) whilst the first stopper component (45) is substantially circular in cross section.

Similarly, the second hollow piston (41) comprises a star shaped orifice (48) and is fitted with a second stopper component (49). The second stopper component (49) is substantially circular in cross section. However, the inner surface (43a) of the side wall (43) is provided with ridges (not shown) which enable the second stopper component (49) to form a seal.

It will be understood by the person skilled in the art that the shape of the orifices (47) and (48) and/or the first and second stopper components (45) and (49) may suitably be varied provided that the stopper (45) and (49) does not form a sealing fit within the orifice (47) and (48).

In use, when the plunger (6) is depressed within the first cartridge (2) the increase in pressure will force the first stopper component (45) out of the first cartridge (2). This movement allows the first pharmaceutical component (7), usually a fluid, to flow from the first cartridge (2) between the stopper component (45) and the side wall (42) of the piston (40) and through first orifice (47) into the second hollow piston (41). Continued depression of the plunger (6) causes the second stopper component (49) to move axially within the second hollow piston (41) away from the ridges (not shown). This movement causes the second stopper component (49) to lose its seal against the side wall (43) of the second hollow piston (41) allowing the first pharmaceutical component (7) to flow from the first cartridge (2) and first hollow piston (40) past the second stopper component (49) and through orifice (48) into the second cartridge (3) where it mixes with the second pharmaceutical component (11).

Referring to FIGS. 5 (a) to (d) a pharmaceutical component-mixing delivery assembly (1) comprises a first cartridge (2) and a second cartridge (3), the first cartridge (2) being dimensioned so that it is slidable within the second cartridge (3) from a pre-mixed position to a post-mixed position. An outer casing (63) is provided which houses the first and second cartridges (2 and 3). The first cartridge (2) comprises a body (4) provided with a sealed outlet end (50). The outer casing (63) comprises a sealed plunger end (51) and the plunger (6) protrudes through the sealed plunger end (51) of the outer casing (63). The first cartridge (2) contains a first pharmaceutical component (7). The second cartridge (3) comprises a body (8) provided with an inlet end (9) and an outlet end (10). The second cartridge (3) contains a second pharmaceutical component (11).

The plunger (6) is provided with a threaded surface (52) such that the threaded surface (52) is present along a predetermined portion of the length of the plunger (6). The sealed plunger end (51) of the outer casing (63) is provided with a stopper (53) which includes an orifice (54) through which the plunger (6) passes and a frangible seal (64). The inner surface (55) of the orifice (54) is provided with a threaded surface (56) which corresponds to and engages with the threaded surface (52) of the plunger (6).

A plug component (57) separates the first and second cartridges (2) and (3). Said plug component (57) comprises a first seal (58) provided with a hollow needle (59) which extends either side of the first seal (58). A second seal (60) comprises a septum (61) provided with a guide channel (62) which, in use, is adapted to receive the needle (59) extending from the first seal (58).

In use, when the plunger (6) is turned within the orifice (54) of the stopper (53) in the outer casing (63) the frangible seal (64) is broken and the plunger (6) and the first cartridge (2) are pushed towards the second cartridge (3), causing the first cartridge (2) to disengage from the frangible seal (64). Continued turning of the plunger causes the first cartridge (2) to slide towards the plug component (57) until the needle (59) pierces the sealed outlet end (50) of the first cartridge (2). Continued turning of the plunger (6) causes the needle (59) to pierce the septum (61) allowing the first pharmaceutical component (7), usually a fluid, to flow from the first cartridge (2) through the needle (59) and into the second cartridge (3) where it mixes with the second pharmaceutical component (11). Once the first and second pharmaceutical components (7) and (11) are mixed and the threaded portion (52) of the plunger (6) has passed through the orifice (54), the plunger (6) may be pushed in a conventional manner to administer the medicament solution to a patient.

Referring to FIGS. 6 and 7 a pharmaceutical component-mixing delivery assembly (1) comprises a first cartridge (2) and a second cartridge (3), the first cartridge (2) being dimensioned so that it is slidable within the second cartridge (3) from a pre-mixed position to a post-mixed position. The first cartridge (2) comprises a body (4) provided with an outlet end (5) and a plunger (6). The first cartridge (2) contains a first pharmaceutical component (7). The second cartridge (3) comprises a body (8) provided with an inlet end (17) and an outlet end (10). The second cartridge (3) contains a second pharmaceutical component (11). A non-invasive valve component (12) separates the first and second cartridges (2) and (3).

The non-invasive valve component (12) comprises a spigot plug component (63) housed in a piston component (64). The piston component (64) is anchored to the first cartridge (2) by a lip (70). The spigot plug component (63) consists of a T-shaped component (65) with a first end (66) of narrower diameter forming a plug in the outlet end (5) of the first cartridge (2) and a second end (67) of wider diameter adjacent the inlet end (17) of the second cartridge (3). The dimensions of the second end (67) are such that it forms an “annular valve” component (68) with the side wall (69) of piston component (64).

In use, when the plunger (6) is depressed within the first cartridge (2) the increase in pressure will force the spigot plug component (63) away from outlet end (5) of the first cartridge (2), which creates a conduit (71) between T-shaped component (65) and the side wall (69) of piston component (64) through which the first pharmaceutical component (7), usually a fluid, can flow into the second cartridge (3) where it mixes with the second pharmaceutical component (11).

Referring to FIGS. 8 and 9 a pharmaceutical component-mixing delivery assembly (1) comprises a first cartridge (2) and a second cartridge (3), the first cartridge (2) being dimensioned so that it is slidable within the second cartridge (3) from a pre-mixed position to a post-mixed position. The first cartridge (2) comprises a body (4) provided with an outlet end (5) and a plunger (6). The first cartridge (2) contains a first pharmaceutical component (7). The second cartridge (3) comprises a body (8) provided with an inlet end (17) and an outlet end (10). The second cartridge (3) contains a second pharmaceutical component (11). A non-invasive valve component (12) separates the first and second cartridges (2) and (3).

The non-invasive valve component (12) comprises a hollow plug component (72) housed in a piston component (73). The piston component (73) is anchored to the first cartridge (2) by a lip (74). The hollow plug component (72) consists of a stopper (75) provided with a T-shaped conduit (76) such that the inlet end (77) of the conduit is coaxial with the first and second cartridges (2 and 3) and the outlet end (78) of the conduit is perpendicular to the inlet end (77) of the conduit, i.e. diametrical to the stopper (75). The piston component (73) is also T-shaped such that the outlet end (79) of the piston component is of greater diameter than the inlet end (80) of the piston component (73). Thus, the outlet end (78) of the conduit is capable of forming an “annular valve” component (81) with the side wall (82) of piston component (73).

In use, when the plunger (6) is depressed within the first cartridge (2) the increase in pressure will force the hollow plug component (72) away from outlet end (5) of the first cartridge (2), which opens the T-shaped conduit (76). Thus, the first pharmaceutical component (7), usually a fluid, can flow through the inlet end (77) of the conduit to the outlet end (78) of the conduit and between the side of the hollow plug component (72) and the side wall (82) of piston component (73) into the second cartridge (3) where it mixes with the second pharmaceutical component (11).

Referring to FIGS. 10 and 11 a pharmaceutical component-mixing delivery assembly (1) comprises a first cartridge (2) and a second cartridge (3), the first cartridge (2) being dimensioned so that it is slidable within the second cartridge (3) from a pre-mixed position to a post-mixed position. The first cartridge (2) comprises a body (4) provided with an outlet end (5) and a plunger (6). The first cartridge (2) contains a first pharmaceutical component (7). The second cartridge (3) comprises a body (8) provided with an inlet end (17) and an outlet end (10). The second cartridge (3) contains a second pharmaceutical component (11). A non-invasive valve component (12) separates the first and second cartridges (2) and (3).

The non-invasive valve component (12) comprises an elongated piston (83). The piston (83) is provided with a plurality of external circumferential ribs (84) which form a seal between the side wall (85) of the piston (83) and the inner wall (86) of the second cartridge (3). The piston is also provided with terminal rib (87), comprising a circumferential rib located at the end (88) of the piston (83) nearest to the inlet end (17) of the second cartridge (3).

The region of the second cartridge (3) that contains the second pharmaceutical component (11) will generally comprise an aseptic mixing chamber (89). The length of the piston (83) will be dictated by how far back along the second cartridge (3) it has to move, which will in turn be dictated by the volume required for the mixing chamber (89). In use, as the plunger (6) is depressed and the piston (83) moves towards the inlet end (17) of the second cartridge (3), the mixing chamber (89) does not come into contact with the non-aseptic inner wall (86) of the inlet end (17) of the second cartridge (3) and the sterility of the mixing chamber (89) is maintained at all times.

1. A pharmaceutical component-mixing delivery assembly comprising:
a first cartridge and a second cartridge;
the first and second cartridges being dimensioned so that they are movable relative to one another such that the first cartridge is slidable within the second cartridge from a pre-mixed position to a post-mixed position;
the first cartridge comprising a body provided with an outlet end, the first cartridge being fitted with plunger and containing a first pharmaceutical component;
the second cartridge comprising a body provided with an inlet end and an outlet end, the second cartridge containing a second pharmaceutical component; and
wherein the first and second cartridges are separated by a non-invasive valve component and the non-invasive valve component is generally elongated, the length of non-invasive valve component being chosen so that, as the mixing chamber expands longitudinally, the pharmaceutical components never come into contact with the non-aseptic walls of the second cartridge thus maintaining sterility at all times.

2. A pharmaceutical component-mixing delivery assembly according to claim 1 wherein the non-invasive valve component is provided with a terminal rib to improve the seal formed between the non-invasive valve component and the second cartridge.

3. A pharmaceutical component-mixing delivery assembly according to claim 1 wherein the non-invasive valve component comprises a ball valve, a flow through stopper component or a T-shaped stopper component.

4. A pharmaceutical component-mixing delivery assembly according to claim 1 wherein the assembly comprises a syringe assembly.

5. A pharmaceutical component-mixing delivery assembly according to claim 4 wherein a hypodermic needle is attached to the outlet end of the second cartridge.

6. A pharmaceutical component-mixing delivery assembly according to claim 1 wherein one of the first and second pharmaceutical components comprises a solid therapeutically active agent and the other is a liquid capable of solubilising the solid therapeutically active agent.

7. (canceled)

8. A pharmaceutical component-mixing delivery assembly according to claim 1 wherein the first pharmaceutical component comprises a liquid and the second pharmaceutical component comprises a liquid.

9-11. (canceled)

12. A pharmaceutical component-mixing delivery assembly according to claim 1 wherein the non-invasive valve component comprises a ball valve.

13. A pharmaceutical component-mixing delivery assembly according to claim 12 wherein the ball valve component comprises one or more balls located within the outlet of the first cartridge, such that the ball(s) acts to seal the first cartridge.

14. A pharmaceutical component-mixing delivery assembly according to claim 13 wherein the ball valve component includes a substantially hollow apertured piston suitable for receiving the ball from the first cartridge.

15. (canceled)

16. A pharmaceutical component-mixing delivery assembly according to claim 12 wherein the ball valve component comprises a plurality of balls.

17. A pharmaceutical component-mixing delivery assembly according to claim 12 wherein the ball valve component includes a plug component which is provided with one or more apertures and which is provided with means for displacing the ball from the outlet of the first cartridge.

18. A pharmaceutical component-mixing delivery assembly according to claim 17 wherein the displacement means comprises a protruding spigot.

19. A pharmaceutical component-mixing delivery assembly according to claim 1 wherein the non-invasive valve component comprises a flow through stopper component.

20-22. (canceled)

23. A pharmaceutical component-mixing delivery assembly according to claim 1 wherein the flow through stopper component is an “annular valve”.

24-28. (canceled)

29. A pharmaceutical component-mixing delivery assembly comprising:
a first cartridge and a second cartridge;
the first and second cartridges being dimensioned so that they are movable relative to one another such that the first cartridge is slidable within the second cartridge from a pre-mixed position to a post-mixed position;
the first cartridge comprising a body provided with an outlet end, the first cartridge being fitted with plunger and containing a first pharmaceutical component;
the second cartridge comprising a body provided with an inlet end and an outlet end, the second cartridge containing a second pharmaceutical component; and wherein the first and second cartridges are separated by an invasive valve component and the plunger is provided with an external screw thread.

30. (canceled)

31. (canceled)

32. A pharmaceutical component-mixing delivery assembly comprising:
an outer casing which houses a first and a second cartridge;
the first and second cartridges being dimensioned so that they are movable relative to one another such that the first cartridge is slidable within the second cartridge from a pre-mixed position to a post-mixed position;
the first cartridge comprising a body provided with an outlet end, the first cartridge being fitted with an at least partially threaded plunger the end of which protrudes from the outer casing, said first cartridge containing a first pharmaceutical component;
the second cartridge comprising a body provided with an inlet end and an outlet end, the second cartridge containing a second pharmaceutical component;
wherein the first and second cartridges are separated by a valve component; and
a frangible seal is provided between the outer casing and the first cartridge.

33. A pharmaceutical component-mixing delivery assembly according to claim 32 wherein the valve component comprises a non-invasive valve.

34. A pharmaceutical component-mixing delivery assembly according to claim 33 wherein the non-invasive valve component is provided with a terminal rib to improve the seal formed between the non-invasive valve component and the second cartridge.

35. A pharmaceutical component-mixing delivery assembly according to claim 32 wherein the valve component may comprise a needle valve.

36. A pharmaceutical component-mixing delivery assembly according to claim 32 wherein the valve component may comprise a bypass valve.

37. (canceled)

38. A method of administering a therapeutically active agent to a patient which comprises operating a pharmaceutical component-mixing delivery assembly holding first and second pharmaceutical components; said delivery assembly comprising:
a first cartridge and a second cartridge;
the first and second cartridges being dimensioned so that they are movable relative to one another such that the first cartridge is slidable within the second cartridge from a pre-mixed position to a post-mixed position;
the first cartridge comprising a body provided with an outlet end, the first cartridge being fitted with plunger and containing a first pharmaceutical component;
the second cartridge comprising a body provided with an inlet end and an outlet end, the second cartridge containing a second pharmaceutical component; and
wherein the first and second cartridges are separated by a non-invasive valve component and the non-invasive valve component is generally elongated, the length of non-invasive valve component being chosen so that as the valve moves, the mixing chamber of the second cartridge never comes into contact with the non-aseptic walls of the second cartridge;
said method comprising the steps of:
(i) pressing the plunger axially into the first cartridge to an intermediate position thus pressurizing the first cartridge and opening the a non-invasive valve component, causing the first pharmaceutical component to flow through the valve and into the second cartridge to mix with the second pharmaceutical component;
(ii) after the first and second pharmaceutical components have mixed, administering the mixed pharmaceutical components to a patient.

39. A method according to claim 38 wherein the non-invasive valve component is provided with a terminal rib to improve the seal formed between the non-invasive valve component and the second cartridge.

40-53. (canceled)
